(19) United States
(12) Patent Application Publication
(10) Pub. No.: US 2009/0326386 A1
(43) Pub. Date: Dec. 31, 2009

(54) Systems and Methods for Non-Invasive Blood Pressure Monitoring

(75) Inventors: Rakesh Sethi, Vancouver (CA); James Watson, Dunfermline (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Galway (IE)

(21) Appl. No.: 12/242,238

(22) Filed: Sep. 30, 2008

Related U.S. Application Data
(60) Provisional application No. 61/076,955, filed on Jun. 30, 2008.
(60) Provisional application No. 61/077,130, filed on Jun. 30, 2008.
(60) Provisional application No. 61/077,132, filed on Jun. 30, 2008.

(51) Int. Cl. A61B 5/021

(52) U.S. Cl. 600/480

(57) ABSTRACT

According to embodiments, systems and methods for non-invasive blood pressure monitoring are disclosed. A sensor or probe may be used to obtain a plethysmograph or photoplethysmograph (PPG) signal from a subject. From the signal, the time difference between two or more characteristic points in the signal may be computed. The time difference may correspond, for example, to the time for a pulse wave to travel a predetermined distance from the senor or probe to a reflection point and back to the sensor or probe. From this time difference, blood pressure measurements may be computed continuously or on a periodic basis.

### CROSS-REFERENCE TO RELATED APPLICATIONS

This claims the benefit of U.S. Provisional Application No. 61/076,955, filed Jun. 30, 2008, 61/077,130, filed Jun. 30, 2008, and 61/077,132, filed Jun. 30, 2008, each of which is hereby incorporated by reference herein in its entirety.

### SUMMARY

The present disclosure relates to blood pressure monitoring and, more particularly, the present disclosure relates to systems and methods for non-invasive blood pressure monitoring.

In some embodiments, a probe or sensor may detect a photoplethysmograph (PPG) signal for use with a continuous non-invasive blood pressure (referred to herein as “CNIBP”) monitoring system or pulse oximeter. The PPG signal may then be analyzed and used to compute a time difference between two or more characteristic points in the detected PPG signal. From this time difference, reliable and accurate blood pressure measurements may be computed on a continuous or periodic basis. Chen et al. U.S. Pat. No. 6,599,251, which is hereby incorporated by reference herein in its entirety, discloses some techniques for continuous and non-invasive blood pressure monitoring using two probes or sensors that may be used in conjunction with the present disclosure.

In some embodiments, the shape of a PPG signal may be considered to be made up of the pulse wave and its many reflections throughout the circulatory system. Because of this consideration, the PPG signal may be useful in determining the blood pressure of a patient by measuring, for example, the time difference between certain characteristic points in the PPG signal. The time difference between the characteristic points in a detected PPG signal may then be used in place of an elapsed time between the arrival of corresponding points of a pulse signal as used in two probe or two sensor CNIBP monitoring techniques.

Characteristics points in the PPG signal may include, for example, the turning points of 1st, 2nd, 3rd (or any other) derivative of the PPG signal, points of inflection in the PPG signal (or in any suitable derivative thereof), stationary points in the PPG signal (or in any suitable derivative thereof), and any suitable peak or valley in the PPG signal and/or in some derivative of the PPG signal. In some embodiments, adjacent peaks (or adjacent valleys) are used as characteristic points in the PPG signal.

From the measured time difference between the two or more characteristics points in the PPG signal, a patient's blood pressure may be monitored continuously or periodically. In addition, in some embodiments, past blood pressure measurements may be used to refine current and future blood pressure measurements. For example, detected blood pressure values outside some pre-defined threshold of a moving average may be ignored in some embodiments. Additionally or alternatively, detected blood pressure values outside of a pre-defined threshold of a moving average may automatically signal a recalibration event. The value of the measured time differences between characteristic points in the PPG signal may also trigger a recalibration event.

A recalibration event may automatically trigger a recalibration sequence. A recalibration sequence may be performed at any suitable time. For example, a recalibration sequence may be performed: 1) initially after device or monitoring initialization; 2) after signaled recalibration events; 3) periodically on a predetermined or other suitable event-driven schedule; 4) at the request of the device user; or 5) at any combination of the aforementioned times. In addition, the characteristic points of the PPG signal used to determine future blood pressure measurements may be varied during (or immediately after) any recalibration sequence in some embodiments. As such, a flexible and adaptive approach may be used in order to improve blood pressure measurements derived from a PPG signal on-the-fly.

Recalibration may be performed, in some embodiments, by measuring a patient's blood pressure (or a reference blood pressure) and then measuring the corresponding elapsed time between a given set of characteristic points in the patient's PPG signal. Updated or refined values for one or more constants or parameters used in the blood pressure measurement determination may then be computed based at least in part on the recalibration. These updated or refined constant or parameter values may then be used to determine the patient's blood pressure until the next recalibration sequence is performed (or for some predetermined length of time).

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative CNIBP monitoring system in accordance with an embodiment;

FIG. 2 is a block diagram of the illustrative CNIBP monitoring system of FIG. 1 coupled to a patient in accordance with an embodiment;

FIG. 3 is a block diagram of an illustrative signal processing system in accordance with some embodiments;

FIG. 4 shows an illustrative PPG signal in accordance with an embodiment;

FIG. 5 shows an illustrative plot tracking systolic and diastolic pressure against a-line data using a single probe in accordance with an embodiment; and

FIG. 6 shows an illustrative process for determining blood pressure in accordance with an embodiment.

### DETAILED DESCRIPTION

Some CNIBP monitoring techniques utilize two probes or sensors positioned at two different locations on a subject's body. The elapsed time, T, between the arrivals of corresponding points of a pulse signal at the two locations may then be determined using signals obtained by the two probes or sensors. The estimated blood pressure, p, may then be related to the elapsed time, T, by

p=a+b·ln(T) (1)

where a and b are constants that may be dependent upon the nature of the subject and the nature of the signal detecting devices. Other suitable equations using an elapsed time between corresponding points of a pulse signal may also be used to derive an estimated blood pressure measurement.

Equation (1) may be used to determine the estimated blood pressure from the time difference, T, between corresponding points of a pulse signal received by two sensors or probes attached to two different locations of a subject. As described in more detail below, however, the value used for the time difference, T, in equation (1) (or in any other blood pressure equation using an elapsed time value between corresponding points of a pulse signal) may also be derived from a signal obtained from a single sensor or probe. In some embodiments, the signal obtained from the single sensor or probe may take the form of a PPG signal obtained, for example, from a CNIBP monitoring system or pulse oximeter.

A PPG signal may be used to determine blood pressure according to the present disclosure at least in part because the shape of the PPG signal may be considered to be made up of the pulse wave and its many reflections throughout the circulatory system. As such, blood pressure equations used in continuous blood pressure monitoring techniques that use sensors or probes at two locations (e.g., equation (1) above) may also be used with continuous blood pressure monitoring techniques that use only a single probe. As described in more detail below, characteristic points may be identified in a detected PPG signal. To determine blood pressure using a PPG signal, the time difference, T, in equation (1) (or in any other blood pressure equation using the time between corresponding points of a pulse signal) may then be substituted with the time between two characteristic points in a detected PPG signal.

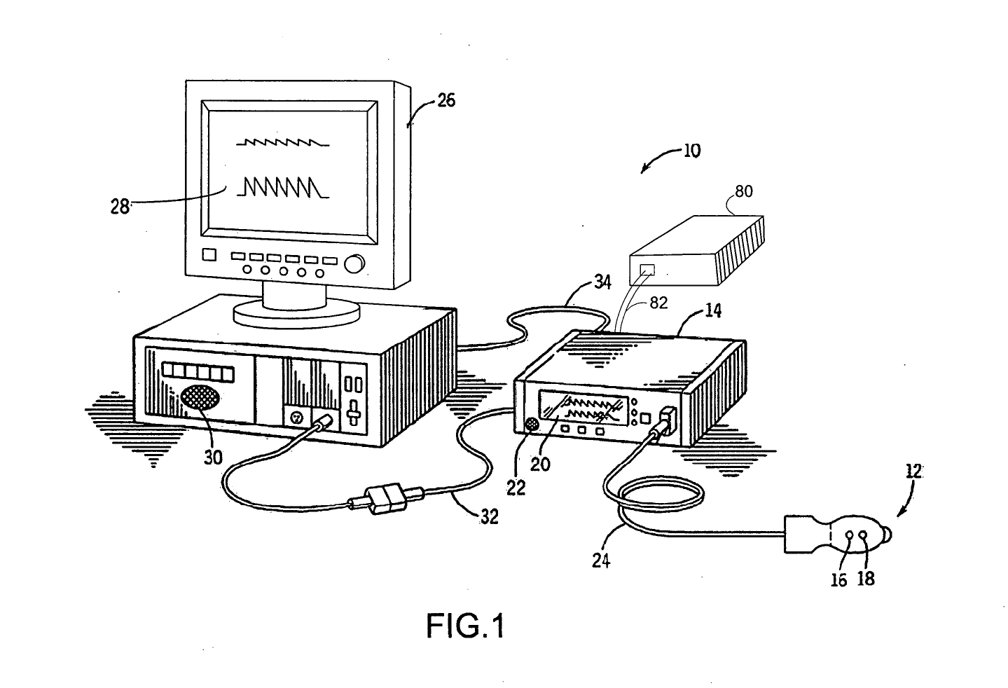

FIG. 1 is a perspective view of an embodiment of a CNIBP monitoring system 10 that may also be used to perform pulse oximetry. System 10 may include a sensor 12 and a monitor 14. Sensor 12 may include an emitter 16 for emitting light at one or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, detector 18 (e.g., a reflective sensor) may be positioned anywhere a strong pulsatile flow may be detected (e.g., over arteries in the neck, wrist, thigh, ankle, ear, or any other suitable location). In an embodiment, emitter 16 and detector 18 may be arranged so that light from emitter 16 penetrates the tissue and is reflected by the tissue into detector 18, such as a sensor designed to obtain pulse oximetry or CNIBP data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., blood pressure) based at least in part on data received from sensor 12 relating to light emission and detection. In an alternative embodiment, the calculations may be performed on the monitoring device itself and the result of the light intensity reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood pressure from monitor 14, blood oxygen saturation generated by monitor 14 (referred to as an “SpO₂” measurement), and pulse rate information from monitor 14.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

Calibration device 80, which may be powered by monitor 14, a battery, or by a conventional power source such as a wall outlet, may include any suitable blood pressure calibration device. For example, calibration device 80 may take the form of any invasive or non-invasive blood pressure monitoring or measuring system used to generate reference blood pressure measurements for use in calibrating the CNIBP monitoring techniques described herein. Such calibration devices may include, for example, an aneroid or mercury sphygmomanometer and occluding cuff, a pressure sensor inserted directly into a suitable artery of a patient, an oscillometric device or any other device or mechanism used to sense, measure, determine, or derive a reference blood pressure measurement. In some embodiments, calibration device 80 may include a manual input device (not shown) used by an operator to manually input reference blood pressure measurements obtained from some other source (e.g., an external invasive or non-invasive blood pressure measurement system).

Calibration device 80 may also access reference blood pressure measurements stored in memory (e.g., RAM, ROM, or a storage device). For example, in some embodiments, calibration device 80 may access reference blood pressure measurements from a relational database stored within calibration device 80, monitor 14, or multi-parameter patient monitor 26. As described in more detail below, the reference blood pressure measurements generated or accessed by calibration device 80 may be updated in real-time, resulting in a continuous source of reference blood pressure measurements for use in continuous or periodic calibration. Alternatively, reference blood pressure measurements generated or accessed by calibration device 80 may be updated periodically, and calibration may be performed on the same periodic cycle. In the depicted embodiments, calibration device 80 is connected to monitor 14 via cable 82. In other embodiments, calibration device 80 may be a stand-alone device that may be in wireless communication with monitor 14. Reference blood pressure measurements may then be wirelessly transmitted to monitor 14 for use in calibration. In still other embodiments, calibration device 80 is completely integrated within monitor 14.

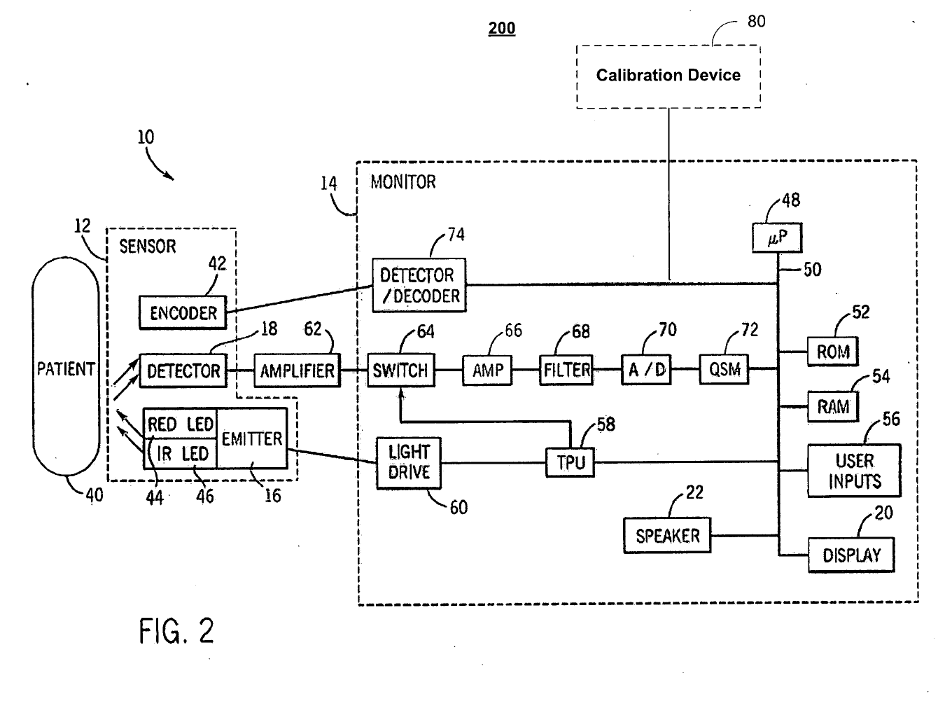

FIG. 2 is a block diagram of a CNIBP monitoring system, such as system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least one wavelength of light (e.g., RED or IR) into a patient's tissue 40. For calculating SpO₂, emitter 16 may include a RED light emitting light source such as RED light emitting diode (LED) 44 and an IR light emitting light source such as IR LED 46 for emitting light into the patient's tissue 40. In other embodiments, emitter 16 may include a light emitting light source of a wavelength other than RED or IR. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the emitted wavelengths (or any other suitable wavelength). Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed, reflected or scattered, less light of that wavelength is received from the tissue by the detector 18. After converting the received light to an electrical signal, detector 18 may send the signal to monitor 14, where physiological parameters may be calculated based on the absorption of one or more of the RED and IR (or other suitable) wavelengths in the patient's tissue 40.

In an embodiment, encoder 42 may contain information about sensor 12, such as what type of sensor it is (e.g., whether the sensor is intended for placement on a forehead or digit) and the wavelength or wavelengths of light emitted by emitter 16. This information may be used by monitor 14 to select appropriate algorithms, lookup tables and/or calibration coefficients stored in monitor 14 for calculating the patients physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelength or wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelength or wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as blood pressure, SpO₂, and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the sensor or probe is attached.

Noise (e.g., from patient movement) can degrade a CNIBP or pulse oximetry signal relied upon by a physician, without the physician's awareness. This is especially true if the monitoring of the patient is remote, the motion is too small to be observed, or the doctor is watching the instrument or other parts of the patient, and not the sensor site. Processing CNIBP or pulse oximetry (i.e., PPG) signals may involve operations that reduce the amount of noise present in the signals or otherwise identify noise components in order to prevent them from affecting measurements of physiological parameters derived from the PPG signals.

CNIBP monitoring system 10 may also include calibration device 80. Although shown external to monitor 14 in the example of FIG. 2, calibration device 80 may additionally or alternatively be internal to monitor 14. Calibration device 80 may be connected to internal bus 50 of monitor 14. As described in more detail below, reference blood pressure measurements from calibration device 80 may be accessed by microprocessor 48 for use in calibrating the CNIBP measurements.

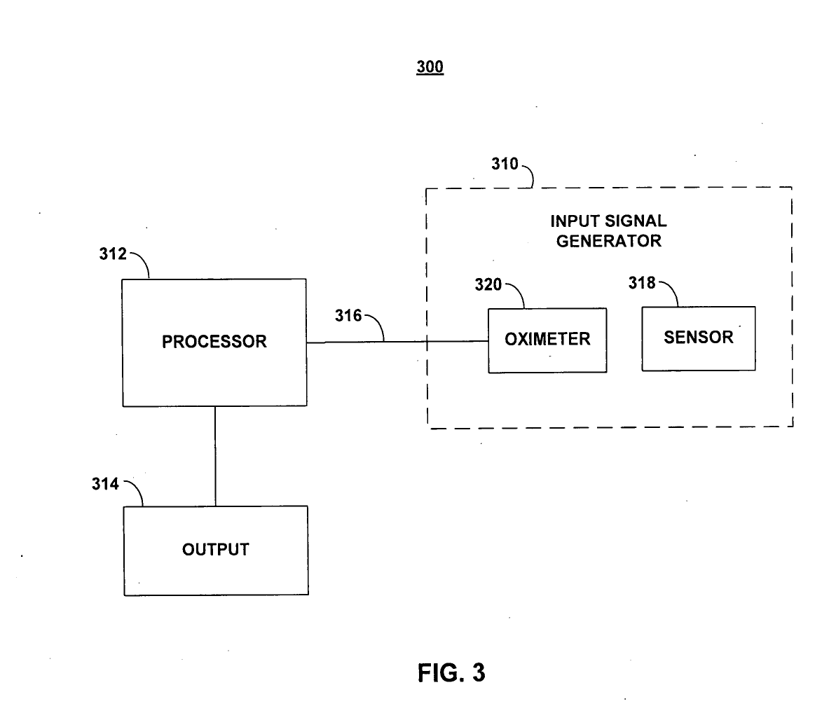

FIG. 3 is an illustrative processing system 300 in accordance with an embodiment. In an embodiment, input signal generator 310 generates an input signal 316. As illustrated, input signal generator 310 may include oximeter 320 (or similar device) coupled to sensor 318, which may provide as input signal 316, a PPG signal. It will be understood that input signal generator 310 may include any suitable signal source, signal generating data, signal generating equipment, or any combination thereof to produce signal 316.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

In an embodiment, signal 316 may be coupled to processor 312. Processor 312 may be any suitable software, firmware, and/or hardware, and/or combinations thereof for processing signal 316. For example, processor 312 may include one or more hardware processors (e.g., integrated circuits), one or more software modules, computer-readable media such as memory, firmware, or any combination thereof. Processor 312 may, for example, be a computer or may be one or more chips (i.e., integrated circuits). Processor 312 may perform some or all of the calculations associated with the blood pressure monitoring methods of the present disclosure. For example, processor 312 may determine the time difference, T, between any two chosen characteristic points of a PPG signal obtained from input signal generator 310. Processor 312 may also be configured to apply equation (1) (or any other blood pressure equation using an elapsed time value) and compute estimated blood pressure measurements on a continuous or periodic basis. Processor 312 may also perform any suitable signal processing of signal 316 to filter signal 316, such as any suitable band-pass filtering, adaptive filtering, closed-loop filtering, and/or any other suitable filtering, and/or any combination thereof. For example, signal 316 may be filtered one or more times prior to or after identifying characteristic points in signal 316.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. Processor 312 may be coupled to a calibration device (not shown) that may generate or receive as input reference blood pressure measurements for use in calibrating CNIBP calculations.

Processor 312 may be coupled to output 314. Output 314 may be any suitable output device such as, for example, one or more medical devices (e.g., a medical monitor that displays various physiological parameters, a medical alarm, or any other suitable medical device that either displays physiological parameters or uses the output of processor 212 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as parts of sensor 12 and monitor 14 and processor 312 may be implemented as pail of monitor 14. In some embodiments, portions of system 300 may be configured to be portable. For example, all or a part of system 300 may be embedded in a small, compact object carried with or attached to the patient (e.g., a watch (or other piece of jewelry) or cellular telephone). In such embodiments, a wireless transceiver (not shown) may also be included in system 300 to enable wireless communication with other components of system 10. As such, system 10 may be part of a fully portable and continuous blood pressure monitoring solution.

According to the present disclosure, reliable blood pressure measurements may be derived from a PPG signal obtained from a single sensor or probe. In some embodiments, the constants a and b in equation (1) above may be determined by performing a calibration. The calibration may involve taking a reference blood pressure reading to obtain a reference blood pressure P₀, measuring the elapsed time T₀ corresponding to the reference blood pressure, and then determining values for both of the constants a and b from the reference blood pressure and elapsed time measurement. Calibration may be performed at any suitable time (e.g., once initially after monitoring begins) or on any suitable schedule (e.g. a periodic or event-driven schedule).

In some embodiments, the calibration may include performing calculations mathematically equivalent to

$$\begin{matrix}a = {c}_{1} + \frac{{c}_{2} ( {P}_{0} - {c}_{1} )}{ln ( {T}_{0} ) + {c}_{2}} \\ and & ( 2 ) \\ b = \frac{{P}_{0} - {c}_{1}}{ln ( {T}_{0} ) + {c}_{2}} & ( 3 )\end{matrix}$$

to obtain values for the constants a and b, where c₁ and c₂ are predetermined constants that may be determined, for example, based on empirical data.

In other embodiments, determining the plurality of constant parameters in the multi-parameter equation (1) may include performing calculations mathematically equivalent to

a=P₀−(c₃T₀+c₄)ln(T₀) (4)

and

b=c₃T₀+c₄ (5)

where a and b are first and second parameters and c₃ and c₄ are predetermined constants that may be determined, for example, based on empirical data.

In some embodiments, the multi-parameter equation (1) may include a non-linear function which is monotonically decreasing and concave upward in a manner specified by the constant parameters.

As mentioned above, multi-parameter equation (1) may be used to determine estimated blood pressure measurements from the time difference, T, between two or more characteristic points of a PPG signal. In some embodiments, the PPG signals used in the CNIBP monitoring techniques described herein are generated by a pulse oximeter or similar device.

The present disclosure may be applied to measuring systolic blood pressure, diastolic blood pressure, mean arterial pressure (MAP) or any combination of the foregoing on an on-going, continuous, or periodic basis. In some embodiments, measuring the time difference, T, includes measuring a first time difference, T_{S}, for certain portions (i.e., portions corresponding generally to the parts of the signals associated with systolic blood pressure) of the PPG signal. Measuring the first time difference may comprise maximizing a cross-correlation between some components of the PPG signal. In such measurements, portions of the PPG signal that fall below a first threshold may not be considered in some embodiments. The first threshold may be an average value for the signal (or equivalently a mean value for the signal).

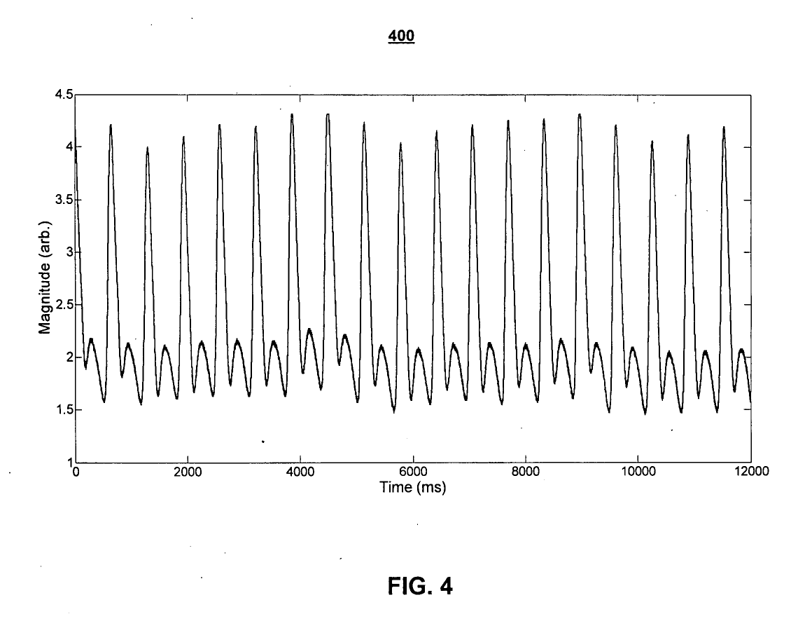

FIG. 4 shows illustrative PPG signal 400. As described above, in some embodiments PPG signal 400 may be generated by a pulse oximeter or similar device positioned at any suitable location of a subject's body. Notably, PPG signal 400 may be generated using only a single sensor or probe attached to the subject's body.

Characteristic points in a PPG (e.g., PPG signal 400) may be identified in a number of ways. For example, in some embodiments, the turning points of 1st, 2nd, 3rd (or any other) derivative of the PPG signal are used as characteristic points. Additionally or alternatively, points of inflection in the PPG signal (or any suitable derivative thereof) may also be used as characteristic points of the PPG signal. The time difference, T, may correspond to the time it takes the pulse wave to travel a predetermined distance (e.g., a distance from the sensor or probe to a reflection point and back to the sensor or probe). Characteristic points in the PPG signal may also include the time between various peaks in the PPG signal and/or in some derivative of the PPG signal, For example, in some embodiments, the time difference, T, may be calculated between (1) the maximum peak of the PPG signal in the time domain and the second peak in the 2nd derivative of the PPG signal (the first 2nd derivative peak may be close to the maximum peak in the time domain) and/or (2) peaks in the 2nd derivative of the PPG signal. Any other suitable time difference between any suitable characteristic points in the PPG signal (e.g., PPG signal 400) or any derivative of the PPG signal may be used as T in other embodiments.

In some embodiments, the time difference between the adjacent peaks in the PPG signal, the time difference between the adjacent valleys in the PPG signal, or the time difference between any combination of peaks and valleys, can be used as the time difference T. As such, adjacent peaks and/or adjacent valleys in the PPG signal (or in any derivative thereof) may also be considered characteristics points. In some embodiments, these time differences may be divided by the actual or estimated heart rate to normalize the time differences. In some embodiments, the resulting time difference values between two peaks may be used to determine the systolic blood pressure, and the resulting time difference values between two valleys may be used to determine the diastolic blood pressure. In an embodiment, the time differences between characteristic points associated with a pulse's maximal and minimal turning points (i.e., those characteristic points associated with maximum and minimum pressures) may be measured from relatively stable points in the PPG signal.

A patient's blood pressure may be monitored continuously using a moving PPG signal. PPG signal detection means may include a pulse oximeter (or other similar device) and associated hardware, software, or both. A processor may continuously analyze the signal from the PPG signal detection means in order to continuously monitor a patient's blood pressure.

In some embodiments, past blood pressure measurements are used to scale current and future measurements. For example, to avoid large swings in detected blood pressure a running or moving blood pressure average may be maintained. Detected blood pressure values outside some pre-defined threshold of the moving average may be ignored in some embodiments. Additionally or alternatively, detected blood pressure values outside some pre-defined threshold of the moving average may automatically signal a recalibration event.

According to some embodiments, one or more calibration (or recalibration) steps may be employed by measuring the patient's blood pressure (or a reference blood pressure), P₀, and then measuring the corresponding elapsed time, T₀, between the chosen characteristic points in the PPG signal. Updated or refined values for constants a and b of equation (1) (or other suitable blood pressure equation) may then be computed based on the calibration. Calibration may be performed once, initially at the start of the continuous monitoring, or calibration may be performed on a regular or event-driven schedule. In some embodiments, calibration may also include changing the characteristic points used to compute the time difference, T. For example, several different blood pressure determinations may be made in parallel using different sets of characteristic points. The set of characteristic points that yields the most accurate blood pressure reading during the calibration period may then be used as the new set of characteristic points. As such, the characteristic points of the PPG signal used in the blood pressure determination may be modified on-the-fly and may vary during a single monitoring session. Such an adaptive approach to selecting characteristic points in the PPG signal may help yield more accurate blood pressure readings.

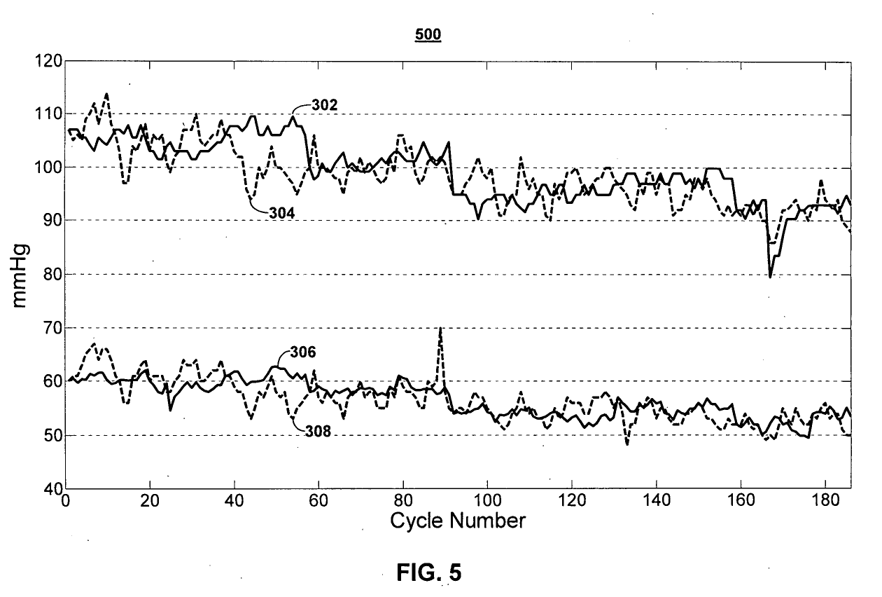

FIG. 5 shows plot 500 tracking systolic and diastolic pressures derived from a PPG signal against a-line data. The a-line data may be derived from, for example, data acquired from a pressure sensor located directly in a suitable artery of a test subject. As such, the a-line data may represent a highly accurate “gold-standard” blood pressure reading. As shown in plot 500, using the blood pressure monitoring techniques described in this disclosure (i.e., blood pressure measurements derived from a PPG signal), systolic blood pressure (line 502) and diastolic blood pressure (line 506) may track the a-line systolic blood pressure (line 504) and the a-line diastolic blood pressure (line 508).

The data illustrated in FIG. 5 may be determined using equation (1) for both diastolic and systolic pressure. To determine systolic pressure, T in equation (1) may be derived, at least in part, from the time difference between the locations of the second maximal turning point of the pulse's second derivative and the pulse's maximum (i.e., peak). Constants a and b may then be derived from equations (4) and (5), respectively. Constants c₂ and c₃ may be derived empirically as −0.4381 and −9.1247, respectively.

To determine diastolic pressure, T in equation (1) may be derived, at least in part, from the time difference between the locations of the second maximal turning point of the pulse's second derivative and the pulse's minimum (i.e., valley). Constants a and b may then be derived from equations (4) and (5), respectively. Constants c₂ and c₃ may be derived empirically as −0.2597 and −4.3789, respectively.

As such, the blood pressure monitoring techniques described in this disclosure may provide a highly accurate and non-invasive solution to measuring a subject's blood pressure.

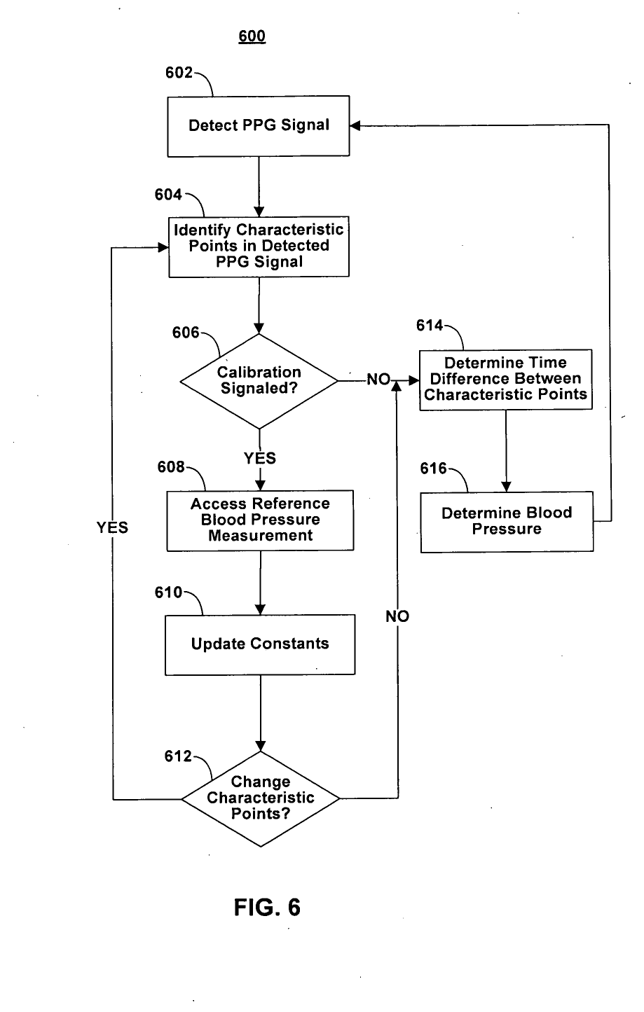

FIG. 6 shows illustrative process 600 for determining blood pressure. At step 602, a PPG signal may be detected from a patient. For example, monitor 14 (FIGS. 1 and 2) may be used to detect a PPG signal from patient 40 (FIG. 2) using, for example, sensor 12 (FIGS. 1 and 2). At step 604, two or more characteristic points may be identified in the detected PPG signal. For example, microprocessor 48 (FIG. 2) may analyze the detected PPG signal and identify various candidate characteristic points in the PPG signal. As described above, peaks, valleys, turning points, and points of inflection in either the PPG signal or any derivative of the PPG signal may be used as suitable characteristic points in some embodiments. Microprocessor 48 (FIG. 2) may identify such characteristic points using any suitable signal processing techniques.

For example, microprocessor 48 (FIG. 2) and/or processor 312 (FIG. 3) may implement various types of digital or analog filtering, using, for example, low pass and band-pass filters in order to preprocess the PPG signal before identifying characteristic points. In some embodiments, to improve results, the PPG signal is first filtered using a low pass or band-pass filter before any derivative of the PPG signal is computed. The signal may be filtered one or more times using any combination of filters.

After the characteristic points are identified in the detected PPG signal, at step 606 a determination is made whether a calibration has been signaled (or should be signaled). As described above, a calibration may be performed once after monitoring initialization or calibration may be performed periodically on any suitable schedule. For example, a calibration event may be signaled by microprocessor 48 (FIG. 2) after blood pressure measurements have exceeded some predefined threshold window or some standard deviation from the mean or moving average of previous measurements. As another example, a calibration event may be signaled by microprocessor 48 (FIG. 2) after the passage of some predetermined length of time from the last calibration event. In such embodiments, microprocessor 48 (FIG. 2) may access a timer or clock and automatically signal calibration events on a periodic schedule.

If calibration has been signaled, at step 608 one or more reference blood pressure measurements may be accessed. For example, calibration device 80 FIGS. 1 and 2) may continuously or periodically generate reference blood pressure measurements for use in calibration. These reference blood pressure measurements may be derived from any suitable invasive or non-invasive blood pressure monitoring technique. The measurements may also be accessed from any suitable storage device, or the measurements may be manually inputted by an operator (e.g., if read from an external monitoring or measurement device).

After the reference blood pressure measurement or measurements are accessed, at step 610 constant parameters may be updated. For example, one or more of constants a and b of equation (1) above may be updated. Any other suitable constants or parameters (of any other suitable blood pressure equation) may be updated in other embodiments. At step 612, a determination is made whether or not to change characteristic points. For example, microprocessor 48 FIG. 2) may dynamically alter the set of characteristic points identified at step 604. In some embodiments, multiple sets of characteristic points are identified in parallel and the set of characteristic points yielding the closest blood pressure measurement to the reference blood pressure measurement accessed at step 608 is selected as the new set of characteristic points.

If a new set of characteristic points are chosen, process 600 may return to step 604 in order to identify the new characteristic points in the detected PPG signal. If the set of characteristic points is not changed at step 612 (or if no calibration is signaled at step 616), then process 600 may continue at step 614. At step 614, the time difference between the identified characteristic points in the PPG signal may be determined. For example, microprocessor 48 (FIG. 2) may compute the time difference between two adjacent peaks, two adjacent valleys, turning points, or points of inflection directly from the detected PPG signal. Microprocessor 48 (FIG. 2) may also compute one or more derivatives of the detected PPG signal and determine the time difference between any two characteristic points in any PPG and derivative signals.

Finally, at step 616, a blood pressure measurement may be determined based, at least in part, on the time difference determined at step 614. For example, equation (1) above (or any other blood pressure equation using an elapsed time between the arrival of corresponding points of a pulse signal) may be used to compute estimated blood pressure measurements. The time difference between characteristic points in the PPG signal may be substituted for the elapsed time between the arrival of corresponding points of a pulse signal. After a blood pressure measurement is determined at step 616, process 600 may return to step 602 and detect a new PPG signal (or access a new segment of a running PPG signal). As such, process 600 may generate blood pressure measurements continuously.

After blood pressure measurements are determined, the measurements may be outputted, stored, or displayed in any suitable fashion. For example, multi-parameter patient monitor 26 (FIG. 1) may display a patient's blood pressure on display 28 (FIG. 1). Additionally or alternatively, the measurements may be saved to memory or a storage device (e.g., ROM 52 or RAM 54 of monitor 14 (FIG. 2)) for later analysis or as a log of a patient's medical history.

In practice, one or more steps shown in process 600 may be combined with other steps, performed in any suitable order, performed in parallel (e.g., simultaneously or substantially simultaneously), or removed.

The foregoing is merely illustrative of the principles of this disclosure and various modifications can be made by those skilled in the art without departing from the scope and spirit of the disclosure. The above described embodiments are presented for purposes of illustration and not of limitation. The present disclosure also can take many forms other than those explicitly described herein. Accordingly, it is emphasized that the disclosure is not limited to the explicitly disclosed methods, systems, and apparatuses, but is intended to include variations to and modifications thereof which are within the spirit of the following claims.

What is claimed is:

1. A method for monitoring blood pressure comprising:
detecting a photoplethysmograph (PPG) signal;
identifying at least two characteristic points in the detected PPG signal;
determining the time difference between two of the at least two characteristic points in the detected PPG signal; and
determining, based at least in part on the determined time difference, a blood pressure measurement.

2. The method of claim 1 wherein identifying at least two characteristic points in the detected PPG signal comprises identifying at least one stationary point or inflection point of at least one derivative of the PPG signal.

3. The method of claim 1 wherein identifying at least two characteristic points in the detected PPG signal comprises identifying a local turning point in the time domain of the PPG signal.

4. The method of claim 1 wherein identifying at least two characteristic points in the detected PPG signal comprises identifying a second peak in a second derivative of the PPG signal.

5. The method of claim 1 wherein identifying at least two characteristic points in the detected PPG signal comprises identifying two peaks in a second derivative of the PPG signal.

6. The method of claim 5 wherein identifying two peaks in the second derivative of the PPG signal comprises identifying two adjacent peaks in the second derivative of the PPG signal.

7. The method of claim 1 wherein determining, based at least in part on the determined time difference, a blood pressure measurement comprises taking a natural logarithm of the time difference.

8. The method of claim 1 wherein determining, based at least in part on the determined time difference, a blood pressure measurement comprises solving a multi-parameter equation that includes the time difference.

9. The method of claim 8 wherein the multi-parameter equation is
p=a+b·ln(T)
or a mathematical equivalent thereof, where p is the determined blood pressure measurement, T is the determined time difference, and a and b are constants.

10. The method of claim 1 further comprising performing at least one calibration of the blood pressure measurement, the calibration based at least in part on a known reference blood pressure measurement.

11. The method of claim 1 further comprising filtering the detected PPG signal one or more times prior to identifying the at least two characteristic points in the detected PPG signal.

12. A system for monitoring blood pressure comprising:
a sensor capable of generating a photoplethysmograph (PPG) signal; and
a processor capable of:
receiving the PPG signal;
identifying at least two characteristic points in the received PPG signal;
determining the time difference between two of the at least two characteristic points in the received PPG signal; and
determining, based at least in part on the determined time difference, a blood pressure measurement.

13. The system of claim 12 wherein the sensor comprises a pulse oximeter.

14. The system of claim 12 wherein the processor is configured to identify at least one stationary point or inflection point of at least one derivative of the PPG signal.

15. The system of claim 12 wherein the processor is configured to identify a local turning point in the time domain of the PPG signal.

16. The system of claim 12 wherein the processor is configured to identify a second peak in a second derivative of the PPG signal.

17. The system of claim 12 wherein the processor is configured to identify two peaks in a second derivative of the PPG signal.

18. The system of claim 17 wherein the processor is configured to identify two adjacent peaks in a second derivative of the PPG signal.

19. The system of claim 12 wherein the processor is configured to determine, based at least in part on the determined time difference, a blood pressure measurement by solving a multi-parameter equation that includes the time difference.

20. The system of claim 19 wherein the multi-parameter equation is
p=a+b·ln(T)
or a mathematical equivalent thereof, where p is the determined blood pressure measurement, T is the determined time difference, and a and b are constants.

21. The system of claim 12 wherein the processor is configured to perform at least one calibration of the blood pressure measurement, the calibration based at least in part on a known reference blood pressure measurement.

22. The system of claim 12 where the processor is configured to filter the detected PPG signal one or more times prior to identifying the at least two characteristic points in the detected PPG signal.

23. A computer-readable medium for use in detecting an artifact in a signal, the computer-readable medium having computer program instructions recorded thereon for:
detecting a photoplethysmograph (PPG) signal;
identifying at least two characteristic points in the detected PPG signal;
determining the time difference between two of the at least two characteristic points in the detected PPG signal; and
determining, based at least in part on the determined time difference, a blood pressure measurement.
